(19) United States
(12) Patent Application Publication
(10) Pub. No.: US 2010/0041747 A1
(43) Pub. Date: Feb. 18, 2010

(54) USE OF CERTAIN CHEMICAL COMPOUNDS FOR THE INHIBITION OF THE PEPTIDYL-PROLYL CIS/TRANS ISOMERASE ACTIVITY OF CYCLOPHILINS

(75) Inventors: Gunter Fischer, Halle (DE); Cordelia Schiene-Fischer, Halle (DE); Sebastian Daum, Halle (DE); Gerhard Küllertz, Halle (DE); Manfred Braun, Schifferstadt (DE)

Correspondence Address: CONNOLLY BOVE LODGE & HUTZ LLP, 1875 EYE STREET, N.W., SUITE 1100, WASHINGTON, DC, 20006, US

(73) Assignees: Max-Planck-Gesellschaft zur Forderung der Wissenschaften e.V., Munchen (DE); Heinrich-Heine-Universitat Dusseldorf, Dusseldorf (DE)

(21) Appl. No.: 12/377,788

(22) PCT Filed: Aug. 14, 2007
(86) PCT No.: PCT/EP2007/007181

§ 371 Date: May 15, 2009

(30) Foreign Application Priority Data
Aug. 17, 2006 (DE) 10 2006 038 403.2

(51) Int. Cl. A61K 31/12; A61K 31/136; A61K 31/365; A61P 29/00; A61P 37/00; A61P 31/04; A61P 31/10; A61P 35/00; A61P 31/12; A61P 33/00; A61P 33/02; A61P 35/02

(52) U.S. Cl. 514/462; 514/677; 514/647; 514/681

(57) ABSTRACT

The present invention relates to the use of certain spiro, ketone and carboxylic acid compounds for the inhibition of the peptidyl-prolyl cis/trans isomerase activity of cyclophilins and the use of these compounds for the production of a cosmetic or pharmaceutical composition for the promotion of hair growth or for the treatment or prevention of inflammatory autoimmune diseases, of diseases caused by fungi, of bacterial infections, of viral infections, of diseases caused by parasites, protozoa or worms, of cancer, of diseases of cells, of fibrosing diseases, and of non-neoplastic changes and diseases which are attributable to prions and changes in the structure and function of cellular proteins and cells.

The present invention relates to the use of certain spiro, ketone and carboxylic acid compounds which have a binding affinity to peptidyl-prolyl cis/trans isomerases of the cyclophilin family, for the inhibition of the peptidyl-prolyl cis/trans isomerase activity of cyclophilins and the use of these compounds for the production of a cosmetic or pharmaceutical composition for the promotion of hair growth or for the treatment or prevention of inflammatory autoimmune diseases, of diseases caused by fungi, of bacterial infections, of viral infections, of diseases caused by parasites, protozoa or worms, of cancer, of diseases of cells, of fibrosing diseases, of non-neoplastic changes and diseases which are attributable to prions and changes in the structure and function of cellular proteins and cells.

The invention is based on the surprising finding that certain spiro, ketone and carboxylic acid compounds inhibit the peptidyl-prolyl cis/trans isomerase (PPIase) activity of cyclophilins, without exerting an immunosuppressive action.

The cyclophilin family belongs to the PPIase class (EC 5.2.1.8). Representatives of this family can be recognized on the basis of their sequence homologies, known to the person skilled in the art, to the representative of the cyclophilins widely distributed in pro- and eukaryotes, cyclophilin 18 (Galat A.: Eur. J. Biochem. 216 (1993) 689-707; Hacker J. & Fischer G.: Mol. Microbiol. 10 (1993) 445-456).

A particular property of the cyclophilins is that their PPIase activity can be inhibited by cyclosporin A (e.g. DD 281659). Cyclophilins in the sense of the present invention should be understood to mean all enzymes which display a PPIase activity which is based on a homology to the known cyclophilins determinable by the usual methods of sequence comparison. Such methods of sequence comparison have been extensively described (e.g. A. Galat: Arch. Bioch. & Biophys. 371 (1999) 149-162; I. T. Chou & C. S. Gasser: Plant Mol. Biol. 35 (1997) 873-892; D. Roy et al.: BBRC 307 (2003) 422-429; J. W. Montague et al.: JBC 272 (1997) 6677-6684). The term cyclophilins is also intended to include those enzymes which as well as cyclophilin amino acid sequences also contain other amino acid sequences attributable to other PPIases, such as for example the enzymes recently described as FCBs (B. Adams et al.: JBC 280 (2005)24308-24314).

As well as the cyclophilins, with the FKBPs and the parvulins there are two further families of PPIases, which can be distinguished by sequence comparison.

For different representatives of the parvulin type of peptidyl-prolyl cis/trans isomerases, the parvulin from Escherichia coli, the ESS1/PTF1 from Saccharomyces cerevisiae and the human Pin1, an irreversible inhibition due to the natural substance juglone (5-hydroxy-1,4-naphthoquinone), specific for parvulins among the prolyl isomerases, could be demonstrated (L. Hennig et al.: Biochemistry 37 (1998) 5953-5960. Juglone is a natural substance with both bacteriostatic and also fungicidal properties, and also cytotoxic properties towards eukaryotic cells (e.g. T. J. Monks et al.: Toxicol Appl Pharmacol 112 (1992) 2-16; N. Didry et al.: Pharmazie 49 (1994) 681-683.

Within the parvulin family, a distinction is made between two groups of enzymes, which differ with regard to their substrate specificity. The first group includes all eukaryotic enzymes with a specificity for (PO₃H₂)Ser/(PO₃H₂)Thr residues in front of the proline in the substrate. These include inter alia the human Pin1 (hPin1) and the ESS1/PTF1 from yeast (e.g. K. P. Lu et al.: Nature 380 (1996) 544-547; S. D. Hanes et al.: Yeast 5 (1998) 55-72; J. Hani et al.: FEBS Lett 365 (1995) 198-202). Previously known prokaryotic and also some of the eukaryotic enzymes are not specific for phosphorylated substrates. They are collected under in the second group.

The reversible phosphorylation of Ser/Thr residues plays a central part in the regulation of fundamental cellular processes. The regulation of the eukaryotic cell cycle is for example subject to the principle of a chronologically very precise sequence of activations of different signal transduction cascades. This process is mainly governed by proline-specific Ser/Thr phosphatases. The reversible phosphorylation of proteins at Ser/Thr residues leads to structural changes of proteins and thereby regulates their biological activity, for example with regard to their stability, enzymatic activity or even their binding affinity towards other proteins (E. A. Nigg: Bioessays 17 (1995) 471-480). The peptide prolyl bond also plays an important part in the definition of the three-dimensional protein structure. It can occur in two different conformations, cis or trans.

Recently a new substance class which can be summarized under the term “spiro, ketone and carboxylic acid derivatives” has been described in WO 03/093258 as inhibitors of phospho-specific parvulins. In the present invention, it could surprisingly be shown that these substances also specifically inhibit the PPIase activity of cyclophilins.

The discovery and the use of inhibitors of the PPIase activity of cyclophilins is of great interest in the scientific literature (e.g. J. F. Guichou et al.: J. Med. Chem. 49 (2006) 900-910; Y. Q. Yu et al.: J. Med. Chem. 46 (2003) 1112-1115) and the object of over a hundred patent specifications (e.g. US 2004204340, US 2003013645, US 2003068321, U.S. Pat. No. 6,270,957, WO 2006033409, WO 2006005580, WO 2005097164 and WO 2005021028).

For many biological effects, in particular the immunosuppressive action of cyclosporin A, the inhibition of the phosphatase activity of the protein phosphatase calcineurin through the complex of cyclosporin A and cyclophilin 18 is regarded as the mechanism of action (e.g. Rusnak F. & Mertz P.: Physiological Reviews 80 (2000) 1483-1521). This mechanism also led in the scientific literature to the term “immunophilins”, which designates PPIases which together with a PPIase inhibitor display immunosuppressive effects (e.g. Dugave C.: Current Organic Chemistry 6 (2002) 1397-1431; Jorgensen K. A. et al.: Scandinavian Journal of Immunology 57 (2003) 93-98). The discovery of the suppressive action of the cyclophilin/cyclosporin A complex on the human immune system led to a range of pharmaceutically important applications in human medicine (e.g.: Pollard S. et al.: Clinical Therapeutics 25 (2003) 1654-1669).

As well as the action of inhibition of the PPIase activity of cyclophilin with cyclosporin A used in transplantation medicine, further effects were also observed, such as for example increased hair growth (e.g. Gafter-Gvili A. et al.: Archives of Dermatological Research 296 (2004) 265-9; Shirai A. et al.: J. of Dermatological Science 27 (2001) 7-13), an effect on hair graying (e.g. Rebora et al.: Int. J. of Dermatology 38 (1999) 229-230), a widely applicable action on mammalian parasites such as for example on the malaria-triggering Plasmodium falciparum (e.g. R. Kumar et al.: Molecular & Biochemical Parasitology 141 (2005) 29-37; A. Bell et al.: Biochemical Pharmacology 48 (1994) 495-503) or on trypanosomes (e.g. J. Bua et al.: Bioorganic and Medicinal Chemistry Letters 14 (2004) 4633-4637), on Leishmania major parasites (e.g. Meissner U. et al.: Parasitology Research 89 (2003) 221-227), on echinococci (e.g. A. L. Colebrook et al.: Parasitology 125 (2002) 485-493) or on nematodes (e.g. D. Ma et al.: JBC 277 (2002) 14925-14932), an action on chlamydia and pneumococci, an action on viruses such as for example on the hepatitis C virus (HCV) or the dengue fever-triggering dengue virus and retroviruses such as the HIV virus (e.g. Boss V. et al.: Molecular Pharmacology 54 (1998) 264-72), an action on psoriasis (e.g. Zachariae H. & Olsen T. S.: Clinical Nephrology 43 (1995) 154-8), an action on nephrotic syndrome (e.g. Noyan A. et al.: Nephron 70 (1995) 410-15) but also therapeutically interesting effects on nerve cells (e.g. Wong A., Cortopassi G.: BBRC 239 (1997) 139-45), pulmonary tissue (e.g. J. W. Eckstein & J. Fung: Expert Opinion on Investigational Drugs 12 (2003) 647-653) and tumors (e.g. T. Z. Wang et al.: Analytical Chemistry 76 (2004) 4343-4348; K. Kawano et al.: Cancer Research 60 (2000) 3550-3558). A large number of these effects were discovered through the use of the active substance cyclosporin A in transplantation medicine and were observed there as a side-effect (e.g. David-Neto E. et al.: J. Am. Soc. Neph. 11 (2000) 343-9).

Particularly serious side-effects of chronic therapy with cyclosporin A can be damage to the kidneys (nephrotoxicity, effects on glomerular filtration, irreversible interstitial fibrosis) e.g. Kopp et al.: J. Am. Soc. Nephrol. 1 (1991) 162-12, neurological changes such as for example tremor (e.g. De Groen et al.: N. Engl. J. Med. 317 (1987) 861-74) and also vascular hypertension (e.g. Kahan et al.: N. Engl. J. Med. 321 (1989) 1725-33), formation of tumors (e.g. Kauffman et al.: Transplantation 80 (2005) 883-889) and complications connected therewith. Also known however are side-effects such as injurious action on the liver or enlargement of the periodontium (e.g. Tosti A. et al.: Drug Safety 10 (1994) 310-17; Borel J. F. et al.: Advances in Pharmacology 35 (1996) 79-114).

More detailed studies of the action of cyclosporin A at the molecular level showed that some of the observed effects can be attributed to the inhibition of the protein phosphatase calcineurin. Through appropriate derivatization of cyclosporin, based on the three-dimensional structure of the cyclosporin A/cyclophilin/calcineurin complex (Jin L. & Harrison S. C.: PNAS 99 (2002) 13522-6), it was subsequently also possible to prepare cyclophilin inhibitors which are able to inhibit the PPIase activity of Cyp18, where however the Cyp18/inhibitor complex is no longer suitable as an inhibitor of the protein phosphatase calcineurin (e.g. Zhang Y. X. et al.: J. of Biological Chemistry 280 (2005) 4842-4850. Such Cyp18 inhibitors not inhibiting the protein phosphatase calcineurin in the complex with Cyp18 are often described as “non-immunosuppressive cyclophilin inhibitors” (e.g. Carry J. C. et al.: Synlett 2 (2004) 316-20; Evers M. et al.: Bioorganic & Medicinal Chemistry Letters 13 (2003) 4415-4419).

However, there are many examples in the literature of low molecular weight substances contributing to immunosuppression even without any inhibition of the protein phosphatase calcineurin. Thus for example PPIase inhibitors such as Sirolimus or Everolimus show comparable effects to cyclosporin A in transplantation immunology, without these substances inhibiting the protein phosphatase calcineurin in vitro (e.g. Lisk W. et al.: Transplantation Proceedings 38 (2006) 69-73). Furthermore, these active substances do not have the cancer triggering action observed with cyclosporin A (e.g. Kauffman et al.: Transplantation 80 (2005) 883-889). While inhibition of calcineurin in combination with an increase in TGFβ leads to cancer progression, it can be shown at least in animals that PPIase inhibitors which are used therapeutically in trans-plantation immunology and do not inhibit calcineurin have an inhibiting effect on tumor growth and tumor angiogenesis (e.g. J. Andrassy et al.: Transplantation 80 (2005) 171-174; S. H. Kim et al.: Am. J. Pathology 164 (2004) 1567-1574; H. Yang et al.: J. of Surgical Res. 123 (2005) 312-319).

Since the peptidyl-prolyl cis/trans isomerase (PPIase) activity of cyclophilins is thus associated with a large number of diseases and cosmetic problems, the purpose of the present invention was to discover substances which inhibit the peptidyl-prolyl cis/trans isomerase (PPIase) activity of cyclophilins.

This problem was solved according to the invention by the use of a compound of the general formula (1)

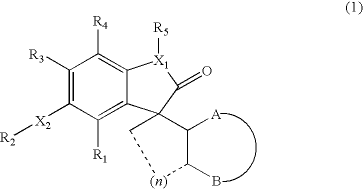

an enantiomer or a preferably pharmaceutically acceptable salt thereof, wherein in the general formula (1)
X₁ is either —CH, —O— or nitrogen;
X₂ is either —CH₂, —O— or —NH—,
R₅ represents no molecular residue under the condition that X₁ is an —O—;
R₂ and R₅ are a hydrogen atom or a straight or branched chain C₁-C₈ alkyl residue, which can be substituted with —OH, —OCH₃, —CH₂CH₂OCH₃, —OCH₂CH₂—N(CH₃)₂ or with NH₂—, C₁-C₈ alkylamino- or with C₁-C₈ dialkylamino groups;
or the unit X₁—R₅ is replaced by O;
R₁, R₃ and R₄ independently of one another are a hydrogen atom, a straight or branched chain C₁-C₈ alkyl residue, a straight or branched chain C₂-C₈ alkenyl residue with one or more double bonds, a straight or branched chain C₂-C₈ alkynyl residue with one or more triple bonds, a halogen atom selected from Cl, Br, I and F, a straight or branched chain C₁-C₈ acyl residue or a C₁-C₈ amidoacyl group of the formula H₂NC(O)— or —HNC(O);
R₃ and R₄ or R₄ and R₅ in addition are together a 5 or 6-membered aromatic or nitrogen, oxygen or sulfur-containing heteroaromatic ring, which can contain F, Cl, Br, I, CN, NO₂, —SH, O and —C(O)H;
(n) together with the dotted lines is a ring which is 4-membered with n=0, 5-membered with n=1, 6-membered with n=2 or 7-membered with n=3, wherein with the exception of the spiro carbon atom each of the ring atoms individually and independently of one another can be a C, N or O atom and these atoms with the exception of oxygen can be linked both by single and also by double bonds, wherein in the case of C as a ring atom this can bear a straight or branched chain C₁-C₈ alkyl residue, a straight or branched chain C₂-C₈ alkenyl residue with one or more double bonds, a straight or branched chain C₂-C₈ alkynyl residue with one or more triple bonds, a keto group, —OH, —OCH₃, —CH₂CH₂OCH₃, OCH₂CH₂—N(CH₃)₂, NH₂—, a C₁-C₈ alkylamino or a C₁-C₈ dialkylamino group, and wherein in the case of N as a ring atom this bears a hydrogen atom or an amino, C₁-C₈ alkylamino or a dialkyamino group;
A and B are either H or a 5 to 7-membered aromatic, heteroaromatic or saturated ring;
or by the use of a compound of the general formula (2)

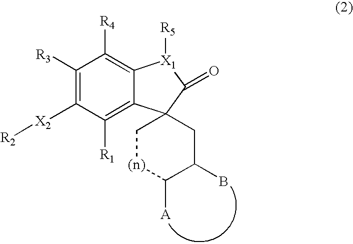

an enantiomer or a preferably pharmaceutically acceptable salt thereof, wherein in the general formula (2) R₁, R₂, R₃, R₄, R₅, X₁, X₂ and A and B respectively are defined as in the general formula (1);
(n) together with the dotted lines is a ring which is 5-membered with n=0, 6-membered with n=1, 7-membered with n=2, or 8-membered with n=3, wherein with the exception of the spiro carbon atom each of the ring atoms individually and independently of one another can be a C, N or O atom and these atoms with the exception of oxygen can be linked both by single and also by double bonds, wherein in the case of C as a ring atom this can bear a straight or branched chain C₁-C₈ alkyl residue, a straight or branched chain C₂-C₈ alkenyl residue with one or more double bonds, a straight or branched chain C₂-C₈ alkynyl residue with one or more triple bonds, a keto group, —OH, —OCH₃, —CH₂CH₂OCH₃, OCH₂CH₂—N(CH₃)₂, NH₂—, a C₁-C₈ alkylamino or a C₁-C₈ dialkylamino group, and wherein in the case of N as a ring atom this can bear a hydrogen atom or an amino, C₁-C₈ alkylamino or a dialkyamino group;
or by the use of a compound of the general formula (3)

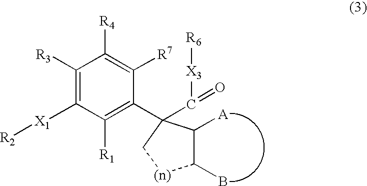

an enantiomer or a preferably pharmaceutically acceptable salt thereof, wherein in the general formula (3)
X₃ is oxygen or NH;

### X₁ is —CH₂, —O— or —NH—;

R₁, R₂, R₃, R₄, (n) and A and B respectively are defined as in the general formula (1);
R₆ and R₇ independently of one another are a hydrogen atom, a straight or branched chain C₁-C₈ alkyl residue, a straight or branched chain C₂-C₈ alkenyl residue with one or more double bonds, a straight or branched chain C₂-C₈ alkynyl residue with one or more triple bonds, a halogen atom selected from Cl, Br, I and F, a straight or branched chain C₁-C₈ acyl residue or a C₁-C₈ amido-acyl group of the formula H₂NC(O)— or —HNC(O);
R₃ and R₄ or R₄ and R₇ in addition can together form a 5 or 6-membered aromatic or heteroaromatic nitrogen, oxygen or sulfur-containing ring, which can bear F, Cl, Br, I, CN, NO₂, —SH, O and/or —C(O)H;
or by the use of a compound of the general formula (4)

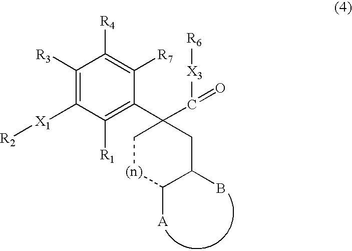

an enantiomer or a preferably pharmaceutically acceptable salt thereof, wherein in the general formula (4) X₁, X₃, R₁, R₂, R₃, R₄, R₆, R₇, and A and B respectively are defined as in the general formula (3) and (n) is respectively defined as in the general formula (2);
or by the use of a compound of the general formula (5)

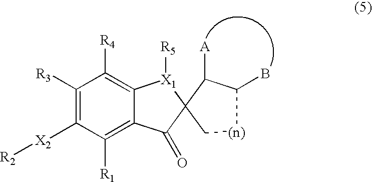

an enantiomer or a preferably pharmaceutically acceptable salt thereof, wherein in the general formula (5) X₁, X₂, R₁, R₂, R₃, R₄, R₅, (n) and A and B respectively are defined as in the general formula (1);
or by the use of a compound of the general formula (6)

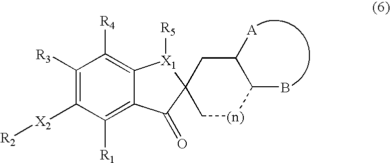

an enantiomer or a preferably pharmaceutically acceptable salt thereof, wherein in the general formula (6) X₁, X₂, R₁, R₂, R₃, R₄, R₅, (n) and A and B respectively are defined as in the general formula (2);
or by the use of a compound of the general formula (7)

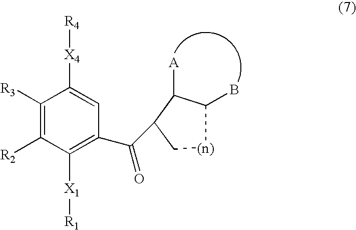

an enantiomer or a preferably pharmaceutically acceptable salt thereof, wherein in the general formula (7)

### X₁ and X₄ are —CH₂—, —O— or —NH—;

R₁ and R₄ are a hydrogen atom or a straight or branched chain C₁-C₈ alkyl residue, which can bear —OH, —OCH₃, —CH₂CH₂OCH₃, OCH₂CH₂—N(CH₃)₂, NH₂—, a C₁-C₈ alkylamino- or a C₁-C₈ dialkylamino group;
R₂ independently thereof is a hydrogen atom, a straight or branched chain C₁-C₈ alkyl residue, a straight or branched chain C₂-C₈ alkenyl residue with one or more double bonds, a straight or branched chain C₂-C₈ alkynyl residue with one or more triple bonds, a halogen atom selected from Cl, Br, I and F, a straight or branched chain C₁-C₈ acyl residue or a C₁-C₈ amidoacyl group of the formula H₂NC(O)— or —HNC(O);
R₃ is appropriately defined as in the general formula (1);
R₂ and R₃ in addition thereto can together form a 5 or 6-membered aromatic or nitrogen, oxygen or sulfur-containing heteroaromatic ring, which can bear F, Cl, Br, I, CN, NO₂, —SH, O and —C(O)H; and
(n) and A and B respectively are defined as in claim 1;
or by the use of a compound of the general formula (8)

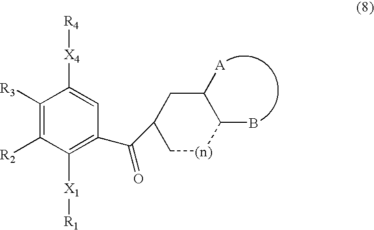

an enantiomer or a preferably pharmaceutically acceptable salt thereof, wherein in the general formula (8) X₁, X₄, R₁, R₂ and R₄ respectively are defined as in the general formula (7) and R₃, (n) and A and B respectively are defined as in the general formula (2);
for the inhibition of the peptidyl-prolyl cis/trans isomerase activity of cyclophilins.

The invention further relates to the use of a compound of the general formula (1), (2), (3), (4), (5), (6), (7) or (8) respectively as defined above for the production of a cosmetic or pharmaceutical composition for the promotion of hair growth or for the treatment or prevention of inflammatory autoimmune diseases, of diseases caused by fungi, of bacterial infections, of viral infections, of diseases caused by parasites, protozoa or worms, of cancer, of diseases of cells, of fibrosing diseases, of non-neoplastic changes and diseases which are attributable to prions and changes in the structure and function of cellular proteins and cells, wherein the said diseases or cosmetic indications are connected with the peptidyl-prolyl cis/trans isomerase activity of cyclophilins.

The invention further relates to the use of a compound of the general formula (1), (2), (3), (4), (5), (6), (7) or (8) respectively as defined above for the production of a cosmetic or pharmaceutical composition for the promotion of hair growth or for the treatment or prevention of hair graying, alopecia, diseases caused by parasites, preferably by malarial plasmodia, trypanosomes, worms, chlamydia or pneumococci, of viral infections, preferably of diseases caused by dengue, AIDS or hepatitis viruses, of nephrotic syndrome, of damage to nerve cells, of diseases of the pulmonary tissue, in particular of pneuomococcal attack, or of diseases attributable to tumor growth and tumor angiogenesis, in particular for the production of a cosmetic or pharmaceutical composition for the promotion of hair growth or for the treatment or prevention of hair graying or alopecia, of diseases caused by parasites, preferably caused by malarial plasmodia, trypanosomes, worms, chlamydia or pneumococci, of viral infections, preferably infections caused by dengue, AIDS or hepatitis viruses.

By means of screening to search for low molecular weight compounds which are able to inhibit cyclophilin 18 (Cyp18), it was possible to find substances which are able to inhibit the PPIase activity of Cyp18, but which, in the complex with Cyp18, are not able to inhibit the protein phosphatase activity of calcineurin towards suitable substrates. An example of a suitable substrate for measurement of the protein phosphatase activity of calcineurin is a phosphorylated peptide derived from a protein substrate and described in the literature as RII peptide (e.g. Baumgrass R. et al.: JBC 276 (2001) 47914-21).

The active substances discovered can be used extremely successfully in vivo in appropriate therapeutic fields of animal and human medicine and also be used ex vivo for appropriate inhibition purposes.

The cyclophilin inhibitors discovered also exert their particular action in the prevention or therapy of a nephrotic syndrome, as also described in the literature for other cyclophilin inhibitors (e.g.: Noyan A. et al.: Nephron 70 (1995) 410-415). Nephrotic syndrome is a collective term and can therefore arise in various diseases of the glomerulus. Nephrotic syndrome arises through increased permeability of the glomerulus to proteins, wherein albumin predominates owing to its high concentration in the plasma. This leads to hypoproteinemia or hypoalbuminemia. In compensation, because of the decreased osmotic pressure, synthesis of the lipoproteins (apoprotein B) increases and at the same time degradation of the VLDL (very low density lipoproteins) decreases, which leads to hypertriglyceridemia. The diseases which can lead to nephrotic syndrome can be divided into two groups: 1) diseases of the kidneys, such as for example minimal change disease, membranous nephropathy and focal segmental glomerular sclerosis; and 2) systemic diseases which can also attack the kidneys, such as for example diabetic nephropathy, amyloidosis, HIV-associated nephropathy, systemic lupus erythematosus and membranoproliferative glomerulonephritis.

The administration of these substances promotes the growth of axons and in particular in the event of neuropathological damage it can bring about the growth, regeneration and repair of nerve cells. This therapeutic action on nerve cells includes the damage to nerve cells caused by general physical attack such as injuries after accidents or surgical interventions. Particularly in diseases which are accompanied by damage to nerve tissue, such as peripheral nerves, motor neurons and the central nervous system (brain and spinal cord), including damage to neurons of the spinal cord and the brain, such as for example in diabetes or stroke or also generally as a result of neurological diseases such as for example Parkinson's or Alzheimer's, the compounds provided are therapeutically successful. An essential property of these therapeutic agents is that these also have prophylactic, protective properties on nerve cells in order to avoid expected or possible damage to nerve cells such as can be necessary for example in stroke prevention or also with intended surgical interventions possibly damaging to nerve cells.

The said damage wherein the substances discovered can advantageously be used therapeutically can also be identified by means of diagnostically ascertainable production or secretion of amyloid β-peptide, also known as abeta or Aβ. Consistently with this, in addition to Parkinson's or Alzheimer's, the substances provided can also be used for the therapy or prevention of tauopathies, prion diseases, frontotemporal dementia, degeneration of the stratum, Lewy body dementia, Huntingdon disease, Pick's disease, and amyloidosis, i.e. all diseases which are associated with excess production of Aβ. Cyclophilin-inhibiting substances which already mention this use field are also already described in the patent literature, for example in WO 2006/005580.

The use of cyclosporin A as a hair restorer has been successfully tested by many researchers both in animal experiments (e.g. Archiv Dermatol. Res. 288 (1996) 408-10) and also in use in man (e.g. J. Am. Acad. Dermatol. 22 (1990) 242-50). Like cyclosporin A, the substances provided can effectively promote the growth of body hair, without inducing the side-effects observed for cyclosporin A, which are probably associated with the inhibition of the protein phosphatase calcineurin. The substances provided can be used both for the treatment of alopecia, in case of excessive hair loss, and also for decreasing the hair loss resulting from chemotherapy.

A further use field is the treatment or prophylaxis of diseases which can be caused by attack by mammalian parasites (protozoa or metazoa). As well as the protozoan and metazoan parasites, viruses and retro-viruses should also be understood to be mammalian parasites. Here, one mode of action of these substances provided is directed at the inhibition of the PPIase activity utilized by these parasites. This inhibited PPIase activity can be both an endogenous PPIase present in the mammalian body, a PPIase only induced in the mammalian body by the parasites, an endogenous PPIase of the parasite itself, and also a mammalian PPIase incorporated in the parasites. In order to obtain comprehensive information concerning the use of substances which are suitable for the treatment or prophylaxis of parasites of mammalian organisms, search systems have been proposed, for example in WO 9511916 (A method for identifying anti-parasitic compounds). Some of these use fields will be described below by way of illustration.

Use in the successful treatment or prophylaxis of viral infections such as for example with retroviruses: the substances discovered can in particular be used as therapeutic agents for AIDS and syndromes associated therewith. Prophylaxis should be understood to mean a treatment of patients in whom the risk of infection with AIDS viruses is very great, or in patients whose AIDS test was positive, but who as yet display no or only slight signs of disease. However those patients in whom treatment with other currently known medicaments leads to improvement or worsening of AIDS symptoms can also be prophylactically treated. The substances discovered can however also be successfully used in order to suppress or reduce the risk of the development of AIDS symptoms in the following months or years after an infection with AIDS-causing retroviruses has taken place. Symptoms of AIDS, such as are for example described by Spirig R.: Advances in Nursing Science 28 (2005) 333-44 or Karus D. et al.: J. of Pain & Symptom Management 30 (2005) 408-17, are well known to the person skilled in the art.

Use in the successful treatment or prophylaxis of viral infections, such as with hepatitis C viruses, which lead to hepatitis C(HCV) disease or infection: in spite of all the scientific advances, such as the cloning of the HCV virus (Choo et al.: Science 244 (1989) 359-362) and the diagnostic detection methods based on this, HCV infection is a major medical problem with ca. 170 million infected persons worldwide at present, since in particular severe liver damage (liver cirrhosis and liver cancer) can occur later as a result of the HCV infection (e.g. Poynard et al.: Lancet 349 (1997) 825-832) and at present no effective protection through vaccination against HCV infection exists (e.g. Manns et al.: Indian J. Gastroenterol. 20 (2001) C47-51). The therapy of choice at present is the combined administration of interferon and ribavirin. This therapy has severe side-effects. Depending on the viral genotype, the therapeutic success rate fluctuates between 90 and 45%. The successful use of cyclophilin inhibitors for HCV therapy has been described several times (e.g.: J. Paeshuyse et al.: Hepatology 43 (2006) 761-770; Nakagawa M. et al.: Gastroenterology 129 (2005) 1031-1041; K. Inoue et al.: J. of Gastroenterology 38 (2003) 567-572).

Corresponding information is also present in patent specifications (e.g. WO 200606038088, WO 200606033409). Since the substances discovered do not cause side-effects caused by calcineurin inhibition, they are particularly suitable for advantageously influencing the HCV infection disadvantageous to the patient by therapeutic administration as a single medicament or together with other medicaments. Typical results can be seen in a decrease in the virus concentration as a function of the duration of therapy with the active substance provided.

There are also indications that active substances which inhibit the PPIase activity of cyclophilin are also able to influence the dengue fever caused by viruses, as described in WO 2004/047759. Dengue fever (dengue hemorrhagic fever (DHF)) can be caused by four different serotypes of the dengue virus. The symptoms are often non-specific or similar to severe influenza, but can also include internal bleeding. Consequently, dengue fever is counted among the hemorrhagic fevers. Dengue fever is also known as seven-day fever, polka fever or breakbone fever.

Use in the successful treatment or prophylaxis of malaria: the substances discovered can in particular be used for the inhibition of the growth of the malaria-causing parasites of the Plasmodium falciparum type in erythrocytes in vitro but also in vivo. Results of extensive studies of the action of cyclophilin inhibitors on the parasites causing malaria are to be found in the literature (e.g. A. Bell: FEMS Microb. Lett. 253 (2005) 171-184; A. Bell et al.: General Pharmacology 27 (1996) 963-971).

Use in the successful treatment or prophylaxis of infections with protozoa in particular Leishmania: the substances discovered can in particular be used for the inhibition of the growth of protozoa such as Leishmania. Results of extensive studies of the action of cyclophilin inhibitors at the molecular level on protozoa are to be found in the literature. The effect of substances inhibiting the PPIase activity of cyclophilin on Leishmania has been thoroughly investigated. Here, the infection of vertebrates with Leishmania occurs by host exchange between insects (e.g. sandflies, moth fly) and vertebrates (e.g. sheep, dogs, man). By means of the substances discovered, a Leishmania infection can be treated irrespective of the species of Leishmania (such as for example: Leishmania aethopica, Leishmania donovani, Leishmania infantum, Leishmania major, Leishmania mexicana, Leishmania amozensis, Leishmania tropica) and irrespective of the disease picture, such as leishmanioses of the skin (skin ulcers, oriental boil), mucosae or viscera.

Use in the successful treatment or prophylaxis of infections with protozoa, in particular trypanosomes, as has already been described for other cyclophilin inhibitors (e.g. J. Bua et al.: Biorganic and Medicinal Chemistry Letters 14 (2004) 4633-4637; U. Meissner et al.: Parasitology Research 89 (2003) 221-227): trypanosomes are the cause of various diseases in mammals. Since trypanosomes evade the immune defenses through their variable proteoglycan coat and a variable modification of proteins by trans-splicing (e.g. T. N. Siegel et al.: Molec. & Cell Biology 25 (2005) 9586-9594), the substances discovered in particular have an advantage over immunotherapy for the treatment and prophylaxis of diseases caused by trypanosomes such as T. brucei brucei (nagana disease in animals), T. brucei gambiensie (African sleeping sickness), T. brucei rhodesensie (East African sleeping sickness) or T. cruzi (Chagas disease).

Use in the successful treatment or prophylaxis of infections with chlamydia: the human pathogenic representatives of these bacteria are Chlamydia pneumoniae which mainly causes pulmonary inflammation and Chlamydia trachomatis which as well as the connective tissue inflammation after which it is named is inter alia responsible for inflammation in the genital region. In Europe, infection with Chlamydia trachomatis is the most frequently occurring sexually transmissible disease of bacterial origin. In Germany, the incidence of Chlamydia trachomatis infection in under age girls is 5.4% (source: Dt. Ärzteblatt, No. 28, 2005). The active substances provided can significantly contribute to the effective therapy of such infections by inhibition of the PPIase activity of the cyclophilin necessary for the infection with chlamydia, as has also been described for the already known cyclophilin inhibitors (e.g. US 2003/037617). Here it is irrelevant whether the cyclophilin required by the chlamydia for the infection is a cyclophilin of the host cell or a bacterial cyclophilin produced by the chlamydia themselves.

Use in the successful treatment or prophylaxis of infections with pneumococci: pneumococci are bacteria which cause severe infections particularly in mammals, small children, elderly people and persons with chronic underlying diseases. Worldwide, about two million people die each year of an infection caused by pneumococci, among them more than a million children under five years with pneumonia. In Germany, about 12,000 people fall victim to a pneumococcal infection each year (e.g. Wikipedia). In spite of antibiotics, half of these deaths already occur within the first 48 hours. As well as pneumonia, pneumococci can cause a large number of other diseases, such as for example meningitis, middle ear inflammation or sinusitis. As is made clear by studies with cyclophilin “knockout” mutants of these bacteria, cyclophilin activity of these bacteria is responsible for bacterial proliferation in the skin cells first affected in an infection (e.g. P. W. M. Herrmans et al.: JBC 281 (2006) 968-976), wherein the cyclophilin used by the pneumococci can be both cyclophilin synthesized by the host cell affected and also by the bacterium itself.

Use in the successful treatment or prophylaxis of infections with helminths (worms), in particular with echinococci, has already been described for other cyclophilin inhibitors (e.g. D. A. Vuitton: Clinical Reviews in Allergy & Immunology 26 (2004) 93-104; A. L. Colebrook et al.: Parasitology 125 (2002) 485-493): here the activity spectrum includes an effect on roundworms (nematodes) and threadworms (filariae). The substances discovered are particularly successful in their action on parasitic worms such as oxyurids, which as a disease cause Enterobius vermicularis, on ascarids with Ascaris lumbricoides (roundworm) as the best known representative, on strongylids with Ankylostoma duodenale or Necator americanus (hookworms), on rhabditids with Strongyloides stercoralis (dwarf threadworm) as representatives, on trichurids with Trichinella spiralis (trichinellae) as the main representative, and trichurids with Trichuris trichiura (Trichocephalus trichiuris, whipworm) as the main representative.

The peptidyl-prolyl cis/trans isomerase activity can, as is described in Practical Example 1, be determined in the so-called protease-coupled PPIase assay by means of isomer-specific proteolysis and correspondingly suitable oligopeptide substrates and isomer-specific proteases. Examples of further methods for PPIase activity determination are the observation of isomer-specific differences with regard to spectroscopic properties, their mobilities or also catalysis of the refolding of other proteins and isomer-specific chemical shifts in the recording of nuclear magnetic resonance spectra. PPIase detection methods are comprehensively documented, for example in G. Fischer & T. Aumuller: Rev. of Physiol. Biochem. & Pharmacol 148 (2004) 105-150; F. Edlich E & G. Fischer G.: Molecular Chaperones in Health and Disease 172 (2006) 350-404; Kullertz et al.: Clin. Chem. 44 (1998) 502-508.

If the addition of active substances to one of the test systems listed above results in a measurable decrease in the rate of reaction catalyzed by the relevant PPIase, these active substances are described as PPIase inhibitors, as is presented in Example 1 by way of example. An assessment of the PPIase specificity is made by the testing of different PPIases in the assay. By variation of the inhibitor concentration and assessment of the measurement results by normal methods, the corresponding inhibition constants (Kᵢ values) can be determined. The inhibitor concentration which 50% inhibits the PPIase-mediated catalysis is described as the IC₅₀ value.

The treatable cancer diseases include hematopoietic changes such as leukemias and lymphomas, and also carcinomas, sarcomas, osteomas, fibrosarcomas, chondrosarcomas and the like. Specific cancer diseases envisaged for treatment with the active substances discovered are mammary cancer, prostate cancer, cervical cancer, stomach cancer, bladder cancer, brain tumors, lung cancer, intestinal cancer, pancreatic cancer, liver cancer, renal cancer and the like.

The said fibrosing diseases also include functional disorders such as fibromyalgia, fibroses (cystic, hepatic, pulmonary, pericardial and the like), fibro-muscular hyperplasia, restenosis, arteriosclerosis and the like.

The compounds usable according to the invention and derivatives thereof are also usable in the treatment of infectious diseases which are due to viruses, bacteria and fungi, and of diseases which are caused by parasitic protozoa.

Among the viral infections that can possibly be treated with the compounds discovered and derivatives thereof are those which are caused by RNA and DNA viruses, such as for example adenoviruses, arboviruses, arenaviruses, bunyaviruses, dengue viruses, flaviviruses, herpes viruses, paramyxoviruses, picornaviruses, polyoma viruses, orbiviruses, orthomyxoviruses, rhabdoviruses, retroviruses, rubella viruses, togaviruses and the like. Inter alia, these diseases include AIDS, hepatitis, encephalitis, meningitis, hemorrhagic fever, colds, hepatitis, blue tongue disease, Colorado tick fever, Lassa fever and Border disease.

The bacterial diseases that can possibly be treated with the compounds discovered include infections caused by gram positive or gram negative bacteria and also by Bacillus, Camphylobacter, Clostridium, Diplococcus, Enterobacter, Enterococcus, Erysipelothricosis, Escherichia, Hemophilus, Klebsiella, Listeria, Morganella, Mycobacterium, Neisseria, Proteus, Providencia, Salmonella, Serratia, Shigella, Staphylococcus, Streptococcus, Yersinia and the like. The said microorganisms cause diseases such as for example brucellosis, cholera, diarrhea, gastroenteritis, gonorrhea, Lyme disease, mastoiditis, meningitis, anthrax, pneumonia, rheumatic fever, dysentery, tetanus, tuberculosis, typhus, and the like.

Among the fungal infections that can possibly be treated with the compounds discovered and derivatives thereof are those which affect either the whole body, the skin or the urogenital tract. Whole body fungal infections are caused by: Absidia, Aspergillus, Candida, Coccidioides, Cryptococcus, Blastocyces, Histoplasma, Hormodendrum, Mucor, Nocardia, Paracoccidioides, Phialopora, Rhinosporidium, Rhizopus, Sporothrix and the like. Skin infections are caused by fungi such as for example Microsporum, Trichophyton, Epidermophyton, Candida and Pityrosporum. Fungal infections affecting the urogenital tract are attributable to fungi such as Aspergillus, Candida, Cryptococcus and Zygomycodoides. All these fungi also cause infectious diseases such as for example dermatophytia, valley fever or San Joaquin fever. These diseases can have serious and fatal consequences, especially in patients with existing immune deficiency, for example after organ transplantation or the presence of AIDS disease (Acquired Immunodeficiency Syndrome).

The compounds discovered and derivatives thereof can also be used for the treatment of parasitic diseases. Causes of these diseases are parasitic protozoa of man, such as Trypanosoma, Leishmania, Trichomonas, Giardia, Entamoeba, Plasmodium, Toxoplasma and Balantidium. Representatives of the above genera cause diseases such as for example sleeping sickness, Chagas disease, trichomonis, forms of dysentery, malaria and toxoplasmosis.

The compounds discovered and derivatives thereof can also be used for the treatment of autoimmune diseases, such as for example psoriasis, neurodermitis, systemic lupus erythematodes, glomerulonephritis, multiple sclerosis, Basedow disease, chronic thyroiditis, myasthenia gravis, pemphigus, scleroderma, ulcerative colitis, rheumatoid arthritis, ITP, hemolytic anemia, diabetes mellitus type I, uveitis, Cogan syndrome and the like.

According to a preferred embodiment of the use according to the invention, the said diseases or cosmetic indications which are associated with the peptidyl-prolyl cis/trans isomerase activity of cyclophilins are caused by the peptidyl-prolyl cis/trans isomerase activity of cyclophilins.

According to a further preferred embodiment of the use according to the invention, the inflammatory autoimmune diseases are selected from the group consisting of psoriasis, neurodermitis, systemic lupus erythematodes, glomerulonephritis, multiple sclerosis, Basedow disease, chronic thyroiditis, myasthenia gravis, pemphigus, scleroderma, ulcerative colitis, rheumatoid arthritis, ITP, hemolytic anemia, diabetes mellitus type I, uveitis and Cogan syndrome.

According to another preferred embodiment of the use according to the invention, the diseases caused by parasites or protozoa are caused by Bacillus, Camphylobacter, Chlamydia, Clostridium, Diplococcus, Enterobacter, Enterococcus, Erysipelothricosis, Escherichia, Hemophilus, Klebsiella, Leishmania, Listeria, Morganella, Mycobacterium, Neisseria, Pneumococci, Proteus, Providencia, Salmonella, Serratia, Shigella, Staphylococcus, Streptococcus, Plasmodium falciparum, trypanosomes or by Yersinia.

According to a further preferred embodiment of the use according to the invention, the viral infections are infections which are caused by viruses selected from the group consisting of adenoviruses, arboviruses, bunyaviruses, dengue viruses, flaviviruses, hepatitis viruses, herpes viruses, paramyxoviruses, picornaviruses, polyoma viruses, orbiviruses, orthomyxoviruses, rhabdoviruses, retroviruses, rubella viruses and togaviruses.

It can be preferred that the diseases caused by fungi affect the whole body, the skin or the urogenital tract and are caused by fungi selected from the group consisting of Absidia, Aspergillus, Candida, Coccidioides, Cryptococcus, Blastocyces, Histoplasma, Hormodendrum, Mucor, Nocardia, Paracoccidioides, Phialopora, Rhinosporidium, Rhizopus, Sporothrix, Microsporum, Trichophyton, Epidermophyton, Candida, Zygomycodoides and Pityrosporum.

Further, it can be preferred that the diseases caused by parasites or protozoa are selected from the group consisting of brucellosis, Chagas disease, cholera, diarrhea, gastroenteritis, gonorrhea, meningeal inflammation, pulmonary inflammation, Lyme disease, malaria, mastoiditis, meningitis, middle ear inflammation, anthrax, sinusitis, nagana disease, sleeping sickness, pneumonias, rheumatic fever, dysentery, tetanus, tuberculosis and typhus.

It can also be preferred that the cancer is a hematopoietic disease, preferably a leukemia or lymphoma, a carcinoma, a sarcoma, an osteoma, a fibrosarcoma or a chondrosarcoma.

According to a preferred embodiment of the use according to the invention, the fibrosing diseases are selected from the group consisting of fibromyalgia, fibroses, fibromuscular hyperplasia, restenosis and arteriosclerosis.

According to a further preferred embodiment of the use according to the invention, the non-neoplastic changes are selected from the group consisting of prostatic hypertrophy, endometriosis and psoriasis.

According to a further embodiment, it is preferred if the diseases which are attributable to prions and changes in the structure and function of cellular proteins and cells are selected from the group consisting of Alzheimer's, Creutzfeld-Jakob disease, and the new variant thereof, nv-Creutzfeld-Jakob disease, scrapie, kuru, fatal familial insomnia and the Gerstmann-Sträussler syndrome. Further, it is preferred if, in the diseases which are attributable to prions and changes in the structure and function of cellular proteins and cells, the cells are nerve cells, preferably nerve cells of peripheral nerves, motor neurons or the central nervous system including the brain and the spinal cord, or the cells have the function of filtering blood.

Furthermore, it can be preferred if the diseases which are attributable to impairment of the perfusion of tissues also include damage which can result in an impairment of perfusion, such as diabetes or stroke, but also those due to neurological diseases such as Parkinson's, Alzheimer's, dementia, Huntington, Pick's disease or impairment of the perfusion of tissues caused by surgical interventions.

It can also be preferred that the diseases caused by worms are attributable to worms (helminths) which affect the whole body or individual organs such as skin, gastrointestinal tract, muscle, liver, lungs or the urogenital tract, or can be caused by echinococci, nematodes, filariae, oxyurids, ascarids, strongylids, rhabditids or trichurids.

It is especially preferred if the diseases which are attributable to tumor growth and tumor angiogenesis are selected from the group consisting of hematopoietic changes, such as leukemias and lymphomas, carcinomas, sarcomas, osteomas, fibrosarcomas, chondrosarcomas and cancer diseases such as for example mammary cancer, prostate cancer, cervical cancer, stomach cancer, bladder cancer, brain tumors, lung cancer, intestinal cancer, pancreatic cancer, liver cancer or renal cancer and other diseases which are characterized by cancer growth.

Further it can also be preferred that the diseases are attributable to morbid changes of the glomerulus and lead to a nephrotic syndrome, are selected from diseases of the kidneys, such as for example minimal change disease, membranous nephropathy and focal segmental glomerular sclerosis, and from systemic diseases which can also affect the kidneys, such as for example diabetic nephropathy, amyloidosis, HIV-associated nephropathy, systemic lupus erythematosus and membranoproliferative glomerulonephritis.

It is also preferred if the compounds according to the general formulae (1), (2), (3), (4), (5), (6), (7) and (8) have a molecular weight of less than 1000 g/mol, preferably less than 750 g/mol and particularly preferably less than 500 g/mol.

In the above general formulae (1) to (8) and below, alkyl means a straight-chain or branched alkyl residue, in particular C₁-C₈ alkyls, such as methyl, ethyl, propyl, isopropyl, butyl, sec-butyl, isobutyl, tert-butyl, n-pentyl, n-hexyl, etc., or an alkyl substituted with aryl, heteroaryl, —O-alkyl, amino, alkylamino or dialkylamino groups, halogen atoms, —OH, CN, NO₂, S, —SR, O and C(O).

An alkenyl can be a straight-chain or branched alkenyl residue, in particular C₂-C₈ alkenyl with one or more double bonds, such as ethene, propene, butene, etc. A substituted alkenyl is an alkenyl substituted with aryl, heteroaryl, halogen atoms, CN, NO₂, S, —SR, O and C(O).

An alkynyl can be a straight-chain or branched alkynyl residue, in particular C₂-C₈ alkenyl with one or more triple bonds. A substituted alkynyl is an alkynyl substituted with aryl, heteroaryl, halogen atoms, CN, NO₂, S, —SR, O and C(O).

Alkylamino means methylamino, ethylamino, propylamino, etc. Dialkylamino is dimethylamino, diethylamino, methylethylamino etc.

Acyl groups can be straight-chain or branched radicals (—R—CO), such as methanoyl, ethanoyl, propanoyl, etc.

In the context of the present invention, multimembered aromatic ring systems are C₃-C₁₃ cyclic systems which can be unsaturated or saturated and be substituted with alkyl and aryl and in the case of heteroaromatic ring systems with heteroaryl, halogen, CN, NO₂, S, O and/or C(O).

Aryl is defined as an organic radical which can have been derived after removal of a hydrogen atom from arenes, i.e. any mono- or polycyclic aromatic and heteroaromatic hydrocarbon compounds; aryl means in particular phenyl. Heteroaryl means in particular five to six-membered aromatics which contain nitrogen, oxygen or sulfur. Aryls and heteroaryls can be aryl substituted with alkyl, aryl, heteroaryl, halogen atoms, CN, NO₂ and C(O).

Halogen means F, Cl, Br or I.

The present invention also relates to the pharmaceutical and cosmetic uses of substances of the stated general formulae and pharmaceutically compatible salts thereof which are coupled to a pharmaceutically acceptable carrier molecule, such as for example amino acids or also oligopeptides.

In a therapy, a therapeutically relevant dose of the compounds should be administered. This is a defined quantity of the compounds which, if administered, leads to the improvement of the symptoms or to prolongation of life for the patient.

The toxicity or the therapeutically effective dose of the compounds discovered can be determined in cell culture assays and animal experiments in the form of LD₅₀ and ED₅₀ values. The LD₅₀ value describes the dose which results in a 50% mortality of a population, while the ED₅₀ value describes the dose which is therapeutically effective in 50% of a population. The ratio between toxicity and therapeutic effect is expressed by the quotient LD₅₀/ED₅₀. Here, naturally, compounds with a high therapeutic potential are preferred. The data which are obtained from the cell culture assays and the animal experiments then serve as the basis for the definition of the doses for the treatment of humans. These preferably lie in the region of the ED₅₀ dose determined, if this is low or not toxic at all. The dose administered here also varies depending on a range of factors, such as for example the presentation of the substance, the mode of administration, the patient's state of health, and the like, and is at the doctor's discretion.

For more exact formulation of the necessary dose, for each of the compounds the therapeutically effective dose can also result from the determination of the IC₅₀ value. Thus, starting from this value a dose present in the circulating plasma of the body, which lies in a concentration range which also corresponds to the IC₅₀ value of the compound in the cell culture assay, can be formulated in animal experiments.

Apart from the definition of the appropriate treatment dose, it is naturally also within the doctor's discretion when the treatment must be ended, interrupted or the dose reduced on account of toxicity, organ disorders inter alia. Likewise, taking account of all the said factors, the doctor can prescribe a dosage increase, if the desired therapeutic effects do not occur. The level of the prescribed dose and the duration of treatment vary depending on various factors, for example the presentation or the nature and severity of the disease. The dose and the duration of treatment also depend on the body weight, age, sex and the reaction of each individual patient to the medicament. Typically, the dose administered varies between 1-50 mg/day/kg body weight. 1-50 mg should be administered to a child and between 25 and 1000 mg to an adult per day.

The compounds according to the formulae (1), (2), (3), (4), (5), (6), (7) and (8) and salts thereof can be administered systemically or topically. Methods and techniques for formulation and administration can be found in “Remington's Pharmaceutical Sciences”. Common administration methods are oral, rectal, vaginal or application onto the skin or mucosae or by means of drug carriers such as for example presentations generally described as plasters, it also being possible to effect the administration onto the skin or mucosa in the form of sprays or by inhalation by means of mist-like finely divided droplets or coupled onto finely dispersed carriers as inhalable dusts. The options for parenteral administration include intramuscular and subcutaneous, and also intrathecal, intraventricular, intravenous, intraperitoneal, intranasal and intraocular injections.

The substances discovered can be formulated as a solution (preferred for parenteral administration) or bound to pharmaceutical carriers (e.g. for oral administration) well known to the person skilled in the art. Coupling onto specific carriers enables the administration of the substance in the form of tablets, pills, capsules, dragees, liquids, gel, granules, syrup, suspensions, slurry and the like.

For parenteral use, the formulation of the substance should be in aqueous solution, and preferably in physiological buffers such as Hank's buffer, Ringer solution or PBS (phosphate-buffered saline). For trans-mucosal injection, the penetration agents normally used and well known to the person skilled in the art can be used. As well as buffer solutions and water, emulsions such as for example oil/water emulsions can also be used. Examples of suitable lipophilic solvents or substances for this are fatty oils such as sesame oil, synthetic fatty acid esters such as ethyl oleate or triglycerides, or liposomes. Suspensions for injection can also contain components which increase the viscosity of the suspension, for example sodium carboxymethylcellulose, sorbitol, dextran and the like. For the preparation of highly concentrated solutions, the solubility of the substances discovered can optionally be increased by means of stabilizers and reagents generally known to the person skilled in the art.

If the intake of the substances discovered is to take place as directly as possible into the cells, this can occur with the aid of liposomes. Liposomes are spherical lipid layers which surround a hydrophilic cavity in which the substances are enclosed. The encapsulated content of the liposomes is thus protected against environmental influences and can efficiently be transferred into the cell after fusion of the lipid layer of the liposomes with the cell membrane of the eukaryotic cell. The mode of action of these lyposomal systems is discussed in the patent publications: International Patent Publication No. WO 91/02805 and International Patent Publication No. WO 91/19501 and in U.S. Pat. No. 4,880,635.

Suitable pharmaceutical carriers for the oral administration of the substances discovered are also familiar to the person skilled in the art and include filler components such as for example lactose, sucrose, mannitol, sorbitol, cellulose components such as maize, wheat, rice and potato starch, gelatin, methyl-cellulose, hydroxypropyl methylcellulose, sodium carboxymethylcellulose, polyvinylpyrrolidine and the like, and mixtures of the said materials. As appropriate, the carrier or the dilution agent can contain known agents for the time-delayed release of the substances, such as glyceryl monostearate or glyceryl distearate alone or in combination with a wax. If necessary, self-disintegrating substances such as agar, crosslinked polyvinylpyrrolidine, alginic acid and the like or salts thereof can be used.

If the substances according to the general formulae (1) to (8) are to be administered as dragees, the concentrated sugar solutions normally used by the person skilled in the art, which for example contain gum arabic, talc, polyvinylpyrrolidone, polyethylene glycol, titanium dioxide, suitable organic solvents or mixtures thereof and the like, can be used for the coating of the tablets. The addition of food dyes and pigments can be used for coloring the dragees and capsules for their better differentiation and for different labeling of different doses.

The following examples in combination with the experimental results shown in the drawing serve to illustrate the invention.

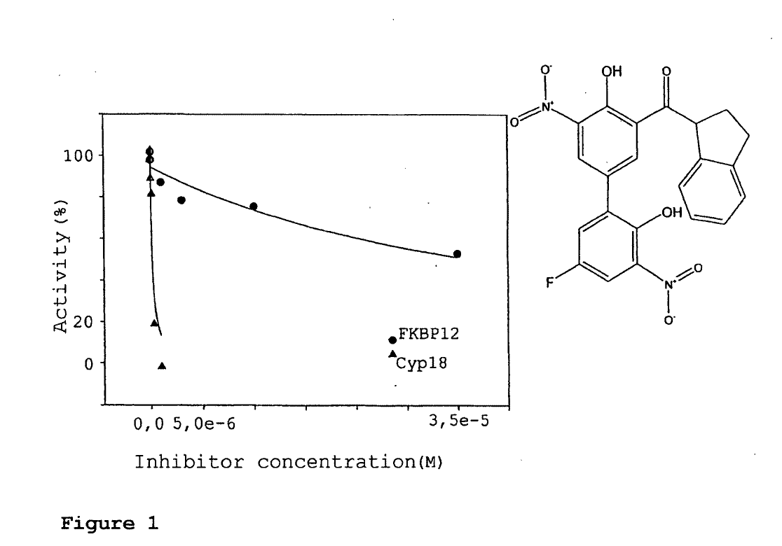

FIG. 1 shows the concentration dependence of the inhibition kinetics of cyclophilin (Cyp18) due to the inhibitor (Br 58) shown in the figure by the molecular formula compared to the corresponding inhibition kinetics of the FKBP family PPIase FKBP12;

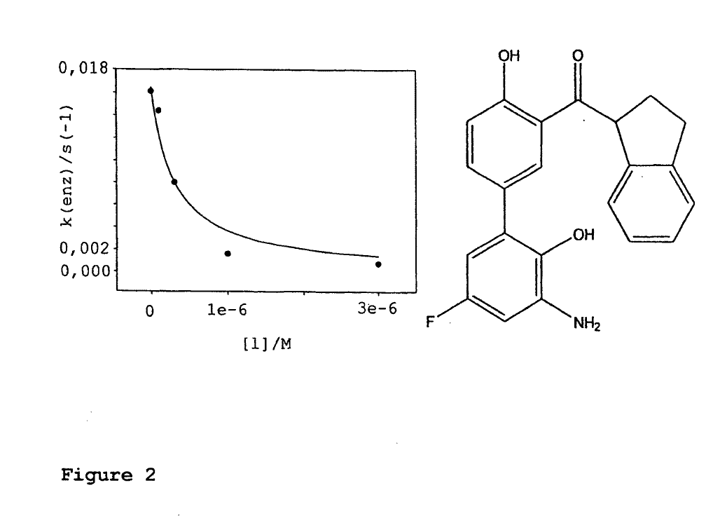

FIG. 2 the concentration dependence of the inhibition kinetics of Cyp18 due to the inhibitor (Br 68) shown in the figure by the molecular formula;

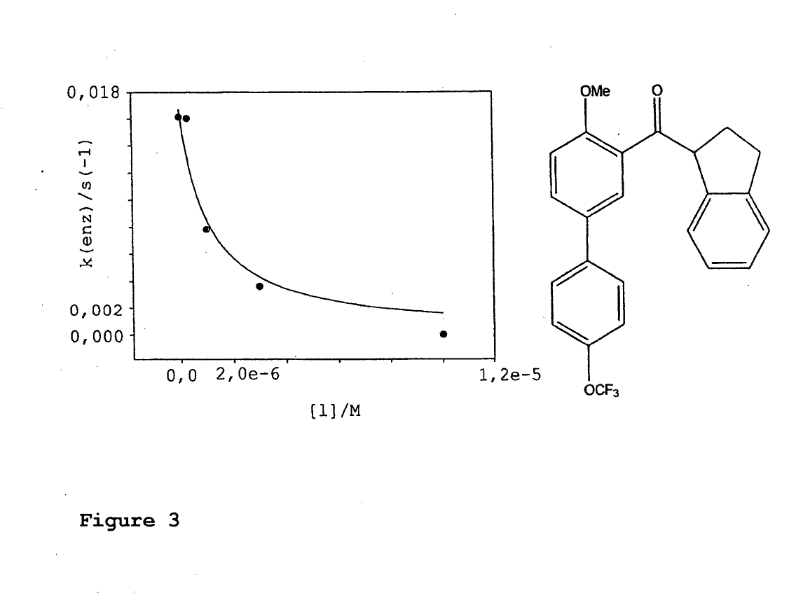

FIG. 3 the concentration dependence of the inhibition kinetics of Cyp18 due to the inhibitor (Br 66) shown in the figure by the molecular formula;

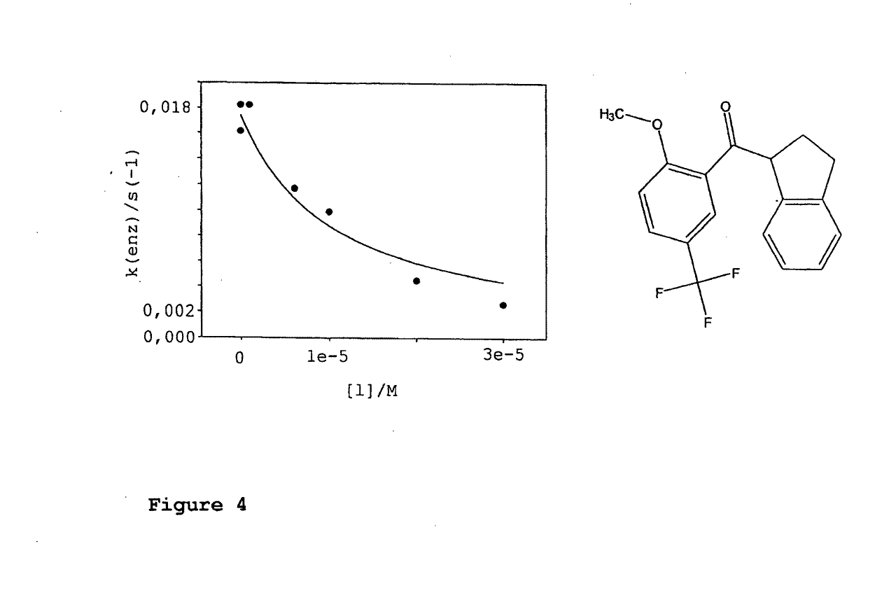

FIG. 4 the concentration dependence of the inhibition kinetics of Cyp18 due to the inhibitor (Br 38) shown in the figure by the molecular formula;

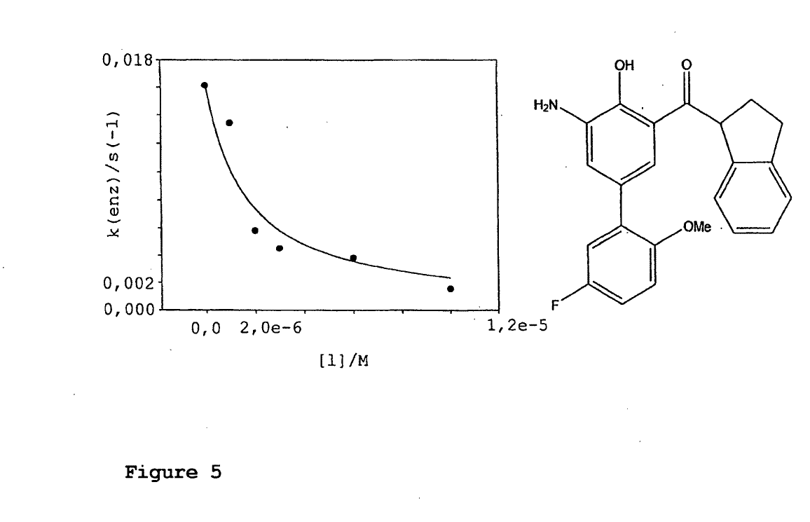

FIG. 5 the concentration dependence of the inhibition kinetics of Cyp18 due to the inhibitor (Br 62) shown in the figure by the molecular formula;

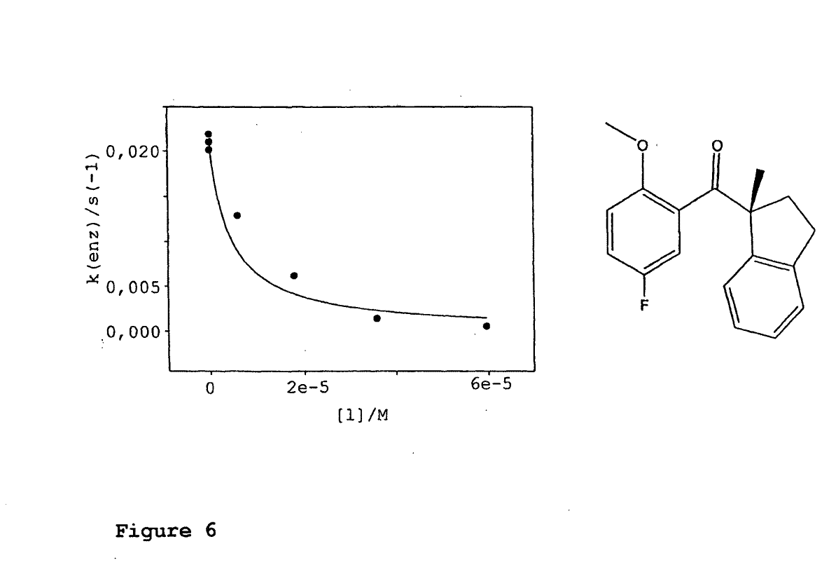

FIG. 6 the concentration dependence of the inhibition kinetics of Cyp18 due to the inhibitor ((−)Br 45) shown in the figure by the molecular formula;

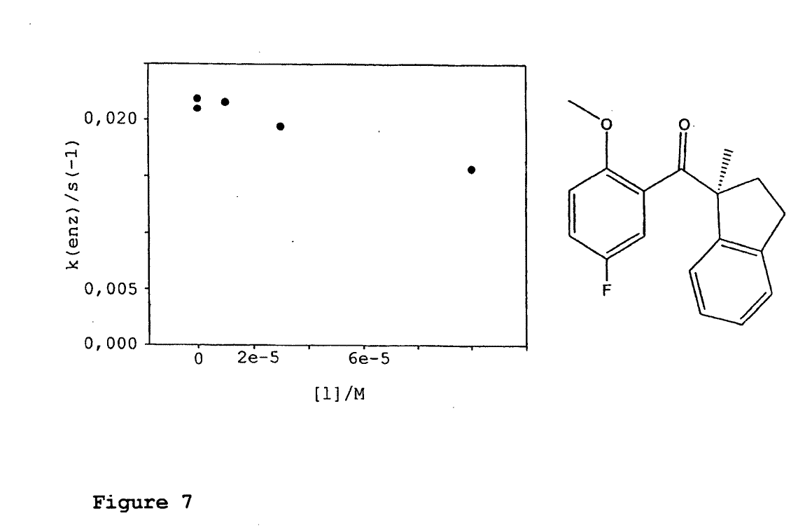

FIG. 7 the concentration dependence of the inhibition kinetics of Cyp18 due to the inhibitor ((+)Br 45) shown in the figure by the molecular formula;

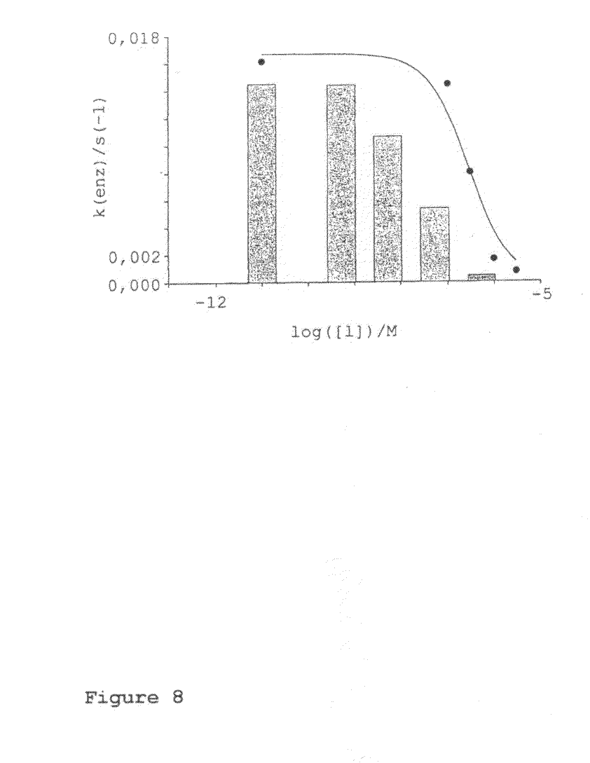

FIG. 8 the demonstration of the reversibility of the cyclophilin inhibition in the case of the inhibitor Br68;

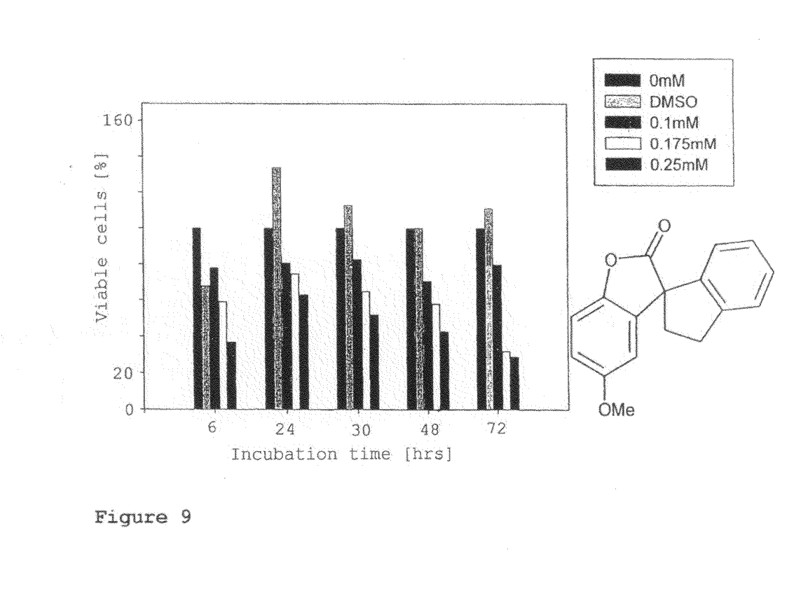

FIG. 9 the determination of the cytotoxicity of 1′,2′,3′H-spiro[inden-3′3-(3H-5-methoxybenzo-furan-2-one)];

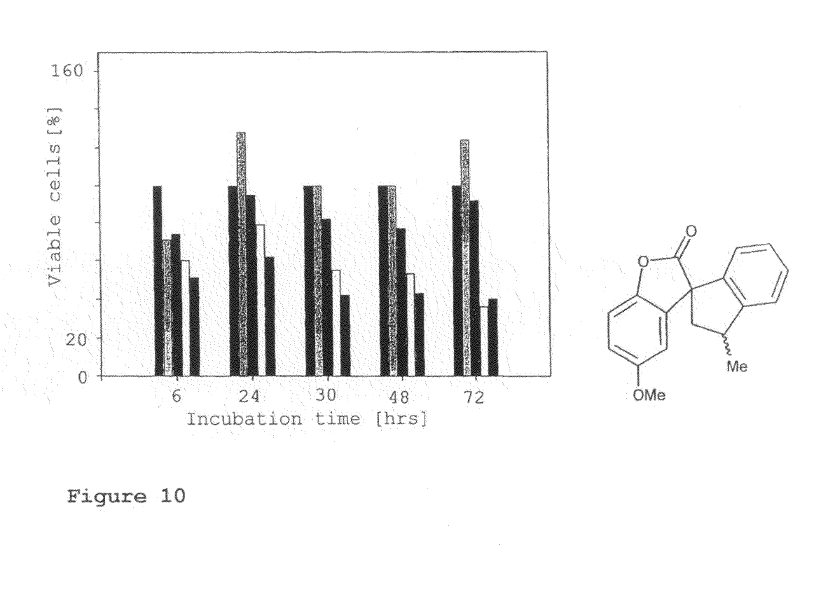

FIG. 10 the determination of the cytotoxicity of 1′,2′,3′H-spiro[1′-methylinden-3′3-(3H-5-methoxy-benzo-furan-2-one)]; and

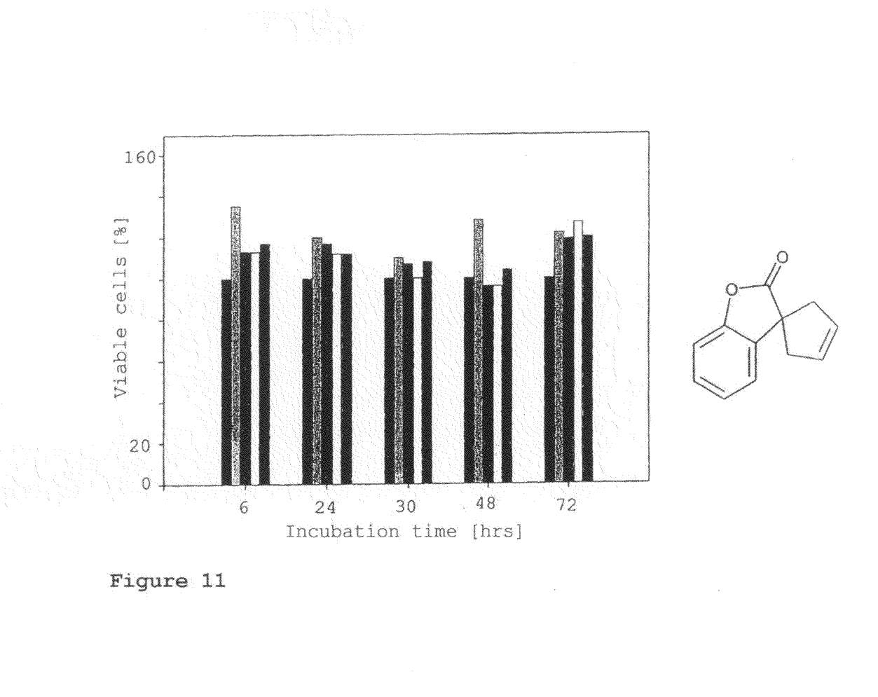

FIG. 11 the determination of the cytotoxicity of 1′,2′,3′H-spiro[cyclopenten-3′3-(3H-5-benzofuran-2-one)].

### EXAMPLE 1

### Determination of Inhibition Constants

### Determination of the Inhibition Constants by Means of Protease-Coupled PPIase Assay

Buffer: 35 mM Hepes (pH 7.8), 1200 μl
Substrate: Ac-Ala-Phe-Pro-Phe-(4-) nitroanilide. Stock solution 10 mg/ml in 35 mM Hepes (pH 7.8)
Auxiliary Chymotrypsin (Merck). Stock solution
protease: 10 mg/ml in 35 mM Hepes (pH 7.8).
   Concentration in assay mixture 0.34 mg/ml.
Enzyme: Human cyclophilin (recombinant from E. coli)
   Stock solution 0.55 μM
   Concentration in assay mixture: 2 nM
Effectors: Effector stock solution 10 mM in DMSO
   Concentration in assay mixture between 1000 and 0.01 μM
Temp.: 10° C.

The effectors were preincubated with the enzyme in the incubation mixture for 5 mins, then 3.5 μl of the auxiliary protease were added and the reaction was then started directly by addition of 3.5 μl of substrate.

The reaction was monitored at a wavelength of 390 nm with the measuring instrument Hewlett Packard UV/VIS spectrophotometer HP 8452A.

The kinetic analysis of the data was performed using “SigmaPlot” (Scientific Graphing System Vers. 2.0, Jandel Corp.).

FIG. 1 shows by way of example the inhibition kinetics for the active substance Br58 in comparison to the almost unaffected activity of the PPIase FKBP12 of the FKBP type.

FIGS. 2 to 7 show the inhibition kinetics for the active substances Br68, Br66, Br38, Br62, (−)Br45 and (+)Br45.

Table 1 shows a compilation of calculated inhibition constants for the enzymes human Pin1, Cyp18 and FKBP12 due to the compounds Br68, Br66, Br38, Br62, (−)Br45 and (+)Br45.

TABLE 1

| | | |
|---|---|---|
| Ki Pin1 | Ki Cyp18 | Ki FKBP12 |

| | | | | | |
|---|---|---|---|---|---|
| Br38 | 4.0 | μM | 10.17 | μM | >100 μM |
| (+)Br45 | 51.29 | μM | >100 | μM | |
| (−)Br45 | 5.63 | μM | 4.46 | μM | |
| Br58 | 246 | nM | 147 | nM | 35.6 μM |
| Br62 | 4.93 | μM | 1.67 | μM | >100 μM |
| Br66 | 1.44 | μM | 1.04 | μM | >100 μM |
| Br68 | 407 | nM | 284 | nM | >100 μM |

### EXAMPLE 2

### Demonstration of the Reversibility of the Cyclophilin Inhibition

The protease-free assay after B. Janowski et al.: Anal. Biochem 252 (1997) 299-307 was used.

Cyclophilin 18 was incubated for 20-30 mins with 20 nM to 200 μM of Br68 and then diluted 1:375. The activities were then measured. The experimental results are shown in FIG. 8.

### EXAMPLE 3

### Cytotoxicity Measurement for Selected Inhibitors

The cytotoxicity of the compounds was determined using the MTT test after Mosmann (1983) [31]. In this, the tetrazolium salt MTT [3-(4,5-dimethylthiazol-2-yl)-2,5-diphenyltetrazolium bromide] is used in a quantitative assay in order to determine the survival or proliferation rate of mammalian cells. Using the assay, living cells were detected. MTT itself is a yellow-brown salt which is cleaved into a deep blue formazan product when it is incubated with living cells with active mitochondria. The reaction that has occurred can then be measured and assessed by spectrophotometry. Here the viability rate of the cells is directly proportional to the absorption of the blue dye at 550 nm.

In the present experiment, 5000 Hela cells were seeded into each well of a 96-well plate and cultured overnight in DMEM (5% FCS, glutamine, antibiotics) to adhere them. Next, fresh medium with the appropriate concentration of the respective substance, dissolved in DMSO (0 mM; 0.1 mM, 0.175 mM or as control the corresponding amount of DMSO in DMEM with 5% FCS and glutamine) was added. The cell viability rate was determined after 6 hrs, 24 hrs, 30 hrs, 48 hrs and 72 hrs. For this, 2.4 mg of MTT were dissolved in 10 ml of serum-free DMEM for 30 mins at 37° C. Next the cell culture medium from the cells was removed from the cells in the microtiter plate, and 100 μl of the MTT medium solution were added to each well. After further incubation for 1 hour at 37° C., the medium was removed and 200 μl DMSO added to each well. The microtiter plate was thoroughly shaken in order to dissolve the cells completely in the DMSO. The viability rate was then determined by measurement of the absorption at 550 and 630 nm on an MR7000 (Dynatech).

The assessment was performed by determination of the absorption difference (Δabs=Abs₅₅₀ₙₘ−Abs₆₃₀ₙₘ). In FIGS. 9, 10 and 11 the experimental results of the cytoxicity measurements on the substances 1′,2′,3′H-spiro[inden-3′3-(3H-5-methoxybenzofuran-2-one)], 1′,2′,3′H-spiro[1′-methylinden-3′3-(3H-5-methoxybenzo-furan-2-one)] and 1′,2′,3′H-spiro[cyclopenten-3′3-(3H-5-benzofuran-2-one)] are shown in graphic form. The mean values of three independent measurement points are given each time in the figures. The values which were obtained for untreated cells were set equal to 100%.

1. A method of inhibiting peptidyl-prolyl cis/trans isomerase activity of cyclophilins comprising administering a compound of the general formula (1) 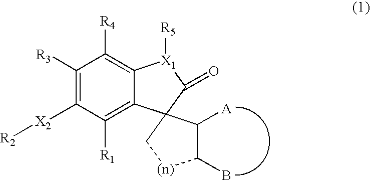
an enantiomer or a preferably pharmaceutically accept-able salt thereof, wherein in the general formula (1)
X₁ is either —CH, —O— or nitrogen;
X₂ is either —CH₂, —O— or —NH—,
R₅ represents no molecular residue under the condition that X₁ is an —O—;
R₂ and R₅ are a hydrogen atom or a straight or branched chain C₁-C₈ alkyl residue, which can be substituted with —OH, —OCH₃, —CH₂CH₂OCH₃, —OCH₂CH₂—N(CH₃)₂ or with NH₂—, C₁-C₈ alkylamino- or with C₁-C₈ dialkylamino groups;
or the unit X₁—R₅ is replaced by O;
R₁, R₃ and R₄ independently of one another are a hydrogen atom, a straight or branched chain C₁-C₈ alkyl residue, a straight or branched chain C₂-C₈ alkenyl residue with one or more double bonds, a straight or branched chain C₂-C₈ alkynyl residue with one or more triple bonds, a halogen atom selected from Cl, Br, I and F, a straight or branched chain C₁-C₈ acyl residue or a C₁-C₈ amidoacyl group of the formula H₂NC(O)— or —HNC(O);
R₃ and R₄ or R₄ and R₅ in addition are together a 5 or 6-membered aromatic or nitrogen, oxygen or sulfur-containing heteroaromatic ring, which can contain F, Cl, Br, I, CN, NO₂, —SH, O and —C(O)H;
(n) together with the dotted lines is a ring which is 4-membered with n=0, 5-membered with n=1, 6-membered with n=2 or 7-membered with n=3, wherein with the exception of the spiro carbon atom each of the ring atoms individually and independently of one another can be a C, N or O atom and these atoms with the exception of oxygen can be linked both by single and also by double bonds, wherein in the case of C as a ring atom this can bear a straight or branched chain C₁-C₈ alkyl residue, a straight or branched chain C₂-C₈ alkenyl residue with one or more double bonds, a straight or branched chain C₂-C₈ alkynyl residue with one or more triple bonds, a keto group, —OH, —OCH₃, —CH₂CH₂OCH₃, OCH₂CH₂—N(CH₃)₂, NH₂—, a C₁-C₈ alkylamino or a C₁-C₈ dialkylamino group, and wherein in the case of N as a ring atom this bears a hydrogen atom or an amino, C₁-C₈ alkylamino or a dialkyamino group; and
A and B are either H or a 5 to 7-membered aromatic, heteroaromatic or saturated ring;
or administering a compound of the general formula (2) 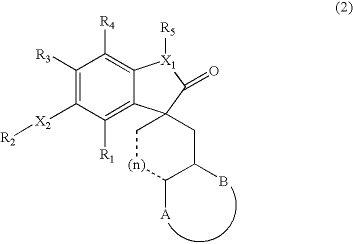
an enantiomer or a preferably pharmaceutically accept-able salt thereof, wherein in the general formula (2) R₁, R₂, R₃, R₄, R₅, X₁, X₂ and A and B respectively are defined as in the general formula (1); and
(n) together with the dotted lines is a ring which is 5-membered with n=0, 6-membered with n=1, 7-membered with n=2, or 8-membered with n=3, wherein with the exception of the spiro carbon atom each of the ring atoms individually and independently of one another can be a C, N or O atom and these atoms with the exception of oxygen can be linked both by single and also by double bonds, wherein in the case of C as a ring atom this can bear a straight or branched chain C₁-C₈ alkyl residue, a straight or branched chain C₂-C₈ alkenyl residue with one or more double bonds, a straight or branched chain C₂-C₈ alkynyl residue with one or more triple bonds, a keto group, —OH, —OCH₃, —CH₂CH₂OCH₃, —OCH₂CH₂—N(CH₃)₂, NH₂—, a C₁-C₈ alkylamino or a C₁-C₈ dialkylamino group, and wherein in the case of N as a ring atom this can bear a hydrogen atom or an amino, C₁-C₈ alkylamino or a dialkyamino group;
or administering a compound of the general formula (3) 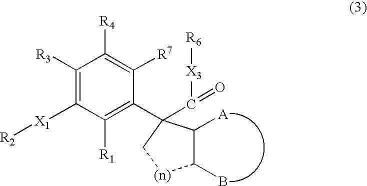
an enantiomer or a preferably pharmaceutically accept-able salt thereof, wherein in the general formula (3)
X₃ is oxygen or NH;
X₁ is —CH₂, —O— or —NH—;
R₁, R₂, R₃, R₄, (n) and A and B respectively are defined as in the general formula (1);
R₆ and R₇ independently of one another are a hydrogen atom, a straight or branched chain C₁-C₈ alkyl residue, a straight or branched chain C₂-C₈ alkenyl residue with one or more double bonds, a straight or branched chain C₂-C₈ alkynyl residue with one or more triple bonds, a halogen atom selected from Cl, Br, I and F, a straight or branched chain C₁-C₈ acyl residue or a C₁-C₈ amido-acyl group of the formula H₂NC(O)— or —HNC(O); and
R₃ and R₄ or R₄ and R₇ in addition can together form a 5 or 6-membered aromatic or heteroaromatic nitrogen, oxygen or sulfur-containing ring, which can bear F, Cl, Br, I, CN, NO₂, —SH, O and/or —C(O)H;
or administering a compound of the general formula (4) 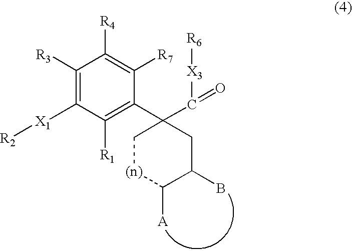
an enantiomer or a preferably pharmaceutically accept-able salt thereof, wherein in the general formula (4) X₁, X₃, R₁, R₂, R₃, R₄, R₆, R₇, and A and B respectively are defined as in the general formula (3) and (n) is respectively defined as in the general formula (2);
or administering a compound of the general formula (5) 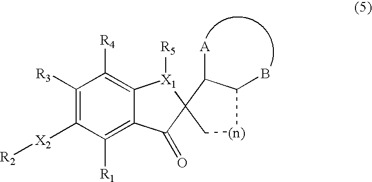
an enantiomer or a preferably pharmaceutically accept-able salt thereof, wherein in the general formula (5) X₁, X₂, R₁, R₂, R₃, R₄, R₅, (n) and A and B respectively are defined as in the general formula (1);
or administering a compound of the general formula (6) 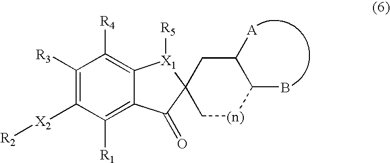
an enantiomer or a preferably pharmaceutically accept-able salt thereof, wherein in the general formula (6) X₁, X₂, R₁, R₂, R₃, R₄, R₅, (n) and A and B respectively are defined as in the general formula (2);
or administering a compound of the general formula (7) 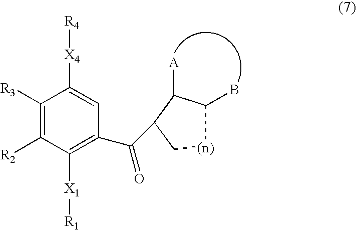
an enantiomer or a preferably pharmaceutically accept-able salt thereof, wherein in the general formula (7)
X₁ and X₄ are —CH₂—, —O— or —NH—;
R₁, and R₄ are a hydrogen atom or a straight or branched chain C₁-C₈ alkyl residue, which can bear —OH, —OCH₃, —CH₂CH₂OCH₃, OCH₂CH₂—N(CH₃)₂, NH₂—, a C₁-C₈ alkylamino- or a C₁-C₈ dialkylamino group;
R₂ independently thereof is a hydrogen atom, a straight or branched chain C₁-C₈ alkyl residue, a straight or branched chain C₂-C₈ alkenyl residue with one or more double bonds, a straight or branched chain C₂-C₈ alkynyl residue with one or more triple bonds, a halogen atom selected from Cl, Br, I and F, a straight or branched chain C₁-C₈ acyl residue or a C₁-C₈ amidoacyl group of the formula H₂NC(O)— or —HNC(O);
R₃ is appropriately defined as in the general formula (1);
R₂ and R₃ in addition thereto can together form a 5 or 6-membered aromatic or nitrogen, oxygen or sulfur-containing heteroaromatic ring, which can bear F, Cl, Br, I, CN, NO₂, —SH, O and —C(O)H; and
(n) and A and B respectively are defined as in claim 1;
or administering a compound of the general formula (8) 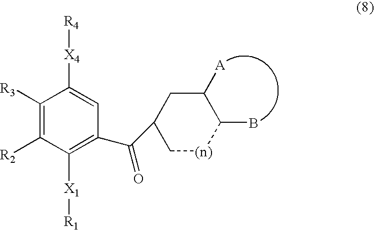
an enantiomer or a preferably pharmaceutically accept-able salt thereof, wherein in the general formula (8) X₁, X₄, R₁, R₂ and R₄ respectively are defined as in the general formula (7) and R₃, (n) and A and B respectively are defined as in the general formula (2).

2. The method as defined in claim 1, wherein the compound of the general formula (1), (2), (3), (4), (5), (6), (7) or (8), respectively, is used for the production of a cosmetic or pharmaceutical composition for the treatment or prevention of inflammatory autoimmune diseases, of diseases caused by fungi, of bacterial infections, of viral infections, of diseases caused by parasites, protozoa or worms, of cancer, of diseases of cells, of fibrosing diseases, of non-neoplastic changes and diseases which are attributable to prions and changes in the structure and function of cellular proteins and cells, wherein the said diseases or cosmetic indications are connected with the peptidyl-prolyl cis/trans isomerase activity of cyclophilins.

3. The method as defined in claim 1, wherein the compound of the general formula (1), (2), (3), (4), (5), (6), (7) or (8), respectively, is used for the production of a cosmetic or pharmaceutical composition for the promotion of hair growth or for the treatment or prevention of hair graying, alopecia, diseases caused by parasites, preferably by malarial plasmodia, trypanosomes, worms, chlamydia or pneumococci, of viral infections, preferably of diseases caused by dengue, AIDS or hepatitis viruses, of nephrotic syndrome, of damage to nerve cells, of diseases of the pulmonary tissue, in particular of pneuomococcal attack, or of diseases attributable to tumor growth and tumor angiogenesis, in particular for the production of a cosmetic or pharmaceutical composition for the promotion of hair growth or for the treatment or prevention of hair graying or alopecia, of diseases caused by parasites, preferably by malarial plasmodia, trypanosomes, worms, chlamydia or pneumococci, and of viral infections, preferably infections caused by dengue, AIDS or hepatitis viruses.

4. The method as claimed in claim 2, wherein the diseases or cosmetic indications are caused by the peptidyl-proline cis/trans isomerase activity of cyclophilins.

5. The method as claimed in claim 2, wherein the inflammatory autoimmune diseases are selected from the group consisting of psoriasis, neurodermitis, systemic lupus erythematodes, glomerulonephritis, multiple sclerosis, Basedow disease, chronic thyroiditis, myasthenia gravis, pemphigus, scleroderma, ulcerative colitis, rheumatoid arthritis, ITP, hemolytic anemia, diabetes mellitus type I, uveitis and Cogan syndrome.

6. The method as claimed in claim 2, wherein the diseases caused by parasites or protozoa are caused by Bacillus, Camphylobacter, Chlamydia, Clostridium, Diplococcus, Enterobacter, Enterococcus, Erysipelo-thricosis, Escherichia, Hemophilus, Klebsiella, Leishmania, Listeria, Morganella, Mycobacterium, Neisseria, Pneumococci, Proteus, Providencia, Salmonella, Serratia, Shigella, Staphylococcus, Streptococcus, Plasmodium falciparum, trypanosomes or by Yersinia.

7. The method as claimed in claim 2, wherein the viral infections are infections which are caused by viruses selected from the group consisting of adenoviruses, arboviruses, bunyaviruses, dengue viruses, flaviviruses, hepatitis viruses, herpes viruses, paramyxoviruses, picornaviruses, polyoma viruses, orbiviruses, orthomyxoviruses, rhabdoviruses, retroviruses, rubella viruses and togaviruses.

8. The method as claimed in claim 2, wherein the diseases caused by fungi affect the whole body, the skin or the urogenital tract and are caused by fungi selected from the group consisting of Absidia, Aspergillus, Candida, Coccidioides, Cryptococcus, Blastocyces, Histoplasma, Hormodendrum, Mucor, Nocardia, Paracoccidioides, Phialopora, Rhinosporidium, Rhizopus, Sporothrix, Microsporum, Trichophyton, Epidermophyton, Zygomycodoides and Pityrosporum.

9. The method as claimed in claim 2, wherein the diseases caused by parasites or protozoa are selected from the group consisting of brucellosis, Chagas disease, cholera, diarrhea, gastroenteritis, gonorrhea, meningeal inflammation, pulmonary inflammation, Lyme disease, malaria, mastoiditis, meningitis, middle ear inflammation, anthrax, sinusitis, nagana disease, sleeping sickness, pneumonia, rheumatic fever, dysentery, tetanus, tuberculosis and typhus.

10. The method as claimed in claim 2, wherein the cancer is a hematopoietic disease, preferably a leukemia or lymphoma, a carcinoma, a sarcoma, an osteoma, a fibrosarcoma or a chondrosarcoma.

11. The method as claimed in claim 2, wherein the fibrosing diseases are selected from the group consisting of fibromyalgia, fibroses, fibro-muscular hyperplasia, restenosis and arteriosclerosis.

12. The method as claimed in claim 2, wherein the non-neoplastic changes are selected from the group consisting of prostatic hypertrophy, endometriosis and psoriasis.

13. The method as claimed in claim 2, wherein the diseases which are attributable to prions and changes in the structure and function of cellular proteins and cells are selected from the group consisting of Alzheimer's, Creutzfeld-Jakob disease, and the new variant thereof, nv-Creutzfeld-Jakob disease, scrapie, kuru, fatal familial insomnia and the Gerstmann-Sträussler syndrome.

14. The method as claimed in claim 2, wherein, in the diseases which are attributable to prions and changes in the structure and function of cellular proteins and cells, the cells are nerve cells, preferably nerve cells of peripheral nerves, motor neurons or the central nervous system including the brain and the spinal cord, or the cells have the function of filtering blood.

15. The method as claimed in claim 2, wherein the diseases which are attributable to impairment of the perfusion of tissues also include damage which can result in an impairment of perfusion, such as diabetes or stroke, but also those due to neurological diseases such as Parkinson's, Alzheimer's, dementia, Huntington, Pick's disease or impairment of the perfusion of tissues caused by surgical interventions.

16. The method as claimed in claim 2, wherein the diseases caused by worms are attributable to worms (helminths) which affect the whole body or individual organs such as skin, gastrointestinal tract, muscle, liver, lungs or the urogenital tract, or can be caused by echinococci, nematodes, filariae, oxyurids, ascarids, strongylids, rhabditids or trichurids.

17. The method as claimed in claim 2, wherein the diseases which are attributable to tumor growth and tumor angiogenesis are selected from the group consisting of hematopoietic changes, such as leukemias and lymphomas, carcinomas, sarcomas, osteomas, fibrosarcomas, chondrosarcomas and cancer diseases such as for example mammary cancer, prostate cancer, cervical cancer, stomach cancer, bladder cancer, brain tumors, lung cancer, intestinal cancer, pancreatic cancer, liver cancer or renal cancer and other diseases which are characterized by cancer growth.

18. The method as claimed in claim 2, wherein the diseases are attributable to morbid changes of the glomerulus and lead to a nephrotic syndrome, are selected from diseases of the kidneys, such as for example minimal change disease, membranous nephropathy and focal segmental glomerular sclerosis, and from systemic diseases which can also affect the kidneys, such as for example diabetic nephropathy, amyloidosis, HIV-associated nephropathy, systemic lupus erythem-atosus and membranoproliferative glomerulonephritis.

19. The method as claimed in claim 2, wherein the compounds according to the general formulae (1), (2), (3), (4), (5), (6), (7) and (8) have a molecular weight of less than 1000 g/mol.
